(19) United States
(12) Patent Application Publication
(10) Pub. No.: US 2023/0330080 A1
(43) Pub. Date: Oct. 19, 2023

(54) THERAPEUTIC TREATMENT USING PROTEIN KINASE C (PKC) INHIBITORS AND CYTOTOXIC AGENTS

(71) Applicant: Cambridge Enterprise Limited, Cambridge, Cambridgeshire (GB)

(72) Inventors: Ingo Ringhausen, Cambridge, Cambridgeshire (GB); Eugene Park, Cambridge, Cambridgeshire (GB); David Cavalla, Cambridge, Cambridgeshire (GB)

(21) Appl. No.: 18/001,655

(22) PCT Filed: Jun. 25, 2021
(86) PCT No.: PCT/GB2021/051621

§ 371 Date: Dec. 13, 2022

(30) Foreign Application Priority Data
Jun. 26, 2020 (GB) 2009764.8

(51) Int. Cl. A61K 31/4545; A61K 31/407; A61K 31/553; A61K 31/4184; A61K 31/635; A61K 31/7076; A61P 35/02

(52) U.S. Cl. CPC A61K 31/4545; A61K 31/407; A61K 31/553; A61K 31/4184; A61K 31/635; A61K 31/7076; A61P 35/02

(57) ABSTRACT

The disclosure provides a PKC inhibitor and a cytotoxic agent for use in therapy, wherein the PKC inhibitor reaches a peak concentration in a subject prior to the cytotoxic agent reaching a peak concentration. The PKC inhibitor and the cytotoxic 5 agent may be used to treat cancer or an autoimmune disease.

### FIELD

This invention relates to the use of Protein Kinase C (PKC) inhibitors in combination with cytotoxic agents for the treatment of diseases, such as cancer and autoimmune disease.

### BACKGROUND

Over the past decade, next generation sequencing technologies have provided opportunities to comprehensively describe the spectrum of genomic abnormalities found in various different B cell malignancies (Chapuy et al., 2018; Puente et al., 2011; Quesada et al., 2012; Schmitz et al., 2018). Ultimately, this has improved our understanding of the underlying genetic mutations contributing to uncontrolled proliferation and extended cell survival while enabling the development of targeted therapies. At the same time, increasing experimental evidence indicates that tumor cells do not survive autonomously, but they require signals derived from the microenvironment to fully unfold their gene mutation-driven pathogenic potential.

Indeed, the composition of the microenvironment has predictive prognostic value for the treatment of patients with follicular lymphoma (Dave et al., 2004) and diffuse large B cell lymphoma (Lenz et al., 2008), underscoring the significance of tumor cell—microenvironment interactions for treatment outcomes. Together, these observations have stimulated the development of targeted therapies interfering with tumor-host interactions.

The introduction of inhibitors targeting kinases downstream of the B cell receptor (BCR) to treat chronic lymphocytic leukemia (CLL) is a recent example of the success of such treatments: In addition to blocking BCR-signals, this class of drugs also affects integrin-mediated adhesion to stromal cells, causing a significant redistribution of malignant B cells from the spleen and lymph nodes to the peripheral blood (de Rooij et al., 2012; Herman et al., 2015). Outside their protective niche, malignant B cells stop proliferating and die. Other approaches to targeting the interactions between malignant B cells and host cells focused on T-cell interactions, druggable with PD-1/PD-Li inhibitors (Xu-Monette et al., 2018) or Lenalidomide (Ramsay et al., 2012), and have demonstrated clinical responses in subsets of patients.

The long-term success of targeted and non-targeted therapies is limited by the genomic instability and rapid evolution of tumor cells, leading to the selection of drug-resistant clones overcoming therapeutic pressure. The selection of BTK mutations in patients treated with Ibrutinib, particularly in cells with dysfunctional p53, illustrates this problem (Ahn et al., 2017). Therefore, it seems desirable to develop therapies that truly target the ability of cells of the microenvironment to support tumor cells. Since there is yet no evidence that these cells evolve through clonal evolution, such therapies may have longer lasting effects before malignant B cells adapt.

Survival signals from mesenchymal stromal cells (MSCs) to malignant B cells depend on protein kinase C-β (PKC-β) function in the microenvironment, where it mediates activation of NF-κB and remodelling of stromal cells. Strikingly, Prkcb deficient (KO) mice were entirely resistant to adoptively transferred tumor cells derived from diseased TCL1-transgenic (tg) mice, whereas Prkcb-wild-type (WT) recipient mice succumbed to a lymphoproliferative disease within a few weeks, underscoring the critical role of the tumor microenvironment for disease progression (Lutzny et al., 2013).

Autoimmune disease is characterised by the presence of auto-reactive immune cells leading to tissue damage (Wang et al., 2015). B cells have been identified as a driving factor in many autoimmune diseases (Martin & Chan, 2004). The role of B cells in autoimmune diseases involves different cellular functions including the well-established secretion of autoantibodies, autoantigen presentation and ensuing reciprocal interactions with T cells, secretion of inflammatory cytokines, and the generation of ectopic germinal centers. Through these mechanisms B cells are involved both in autoimmune diseases that are traditionally viewed as antibody mediated and also in autoimmune diseases that are commonly classified as T cell mediated (Hampe, 2012).

B cell depletion has been shown to be beneficial in various autoimmune disorders (Hofmann et al., 2018). The immunosuppressive quality of chemotherapeutics means they are useful in the treatment of autoimmune disease. Examples include, Cyclophosphamide (an alkylating agent) in the treatment of multiple sclerosis (Makhani et al., 2009; Gladstone at al., 2006), Rituximab (an anti-CD20 antibody) in the treatment of systemic lupus erythematosus, Sjögren's syndrome and Grave's disease (Ramos-Casals et al., 2008, El Fassi et al., 2007), and Methotrexate (an antimetabolite) in the treatment of rheumatoid arthritis (St. Clair et al., 2004). There remains a need for improved therapies that interfere with host interactions with disease cells, such as auto-reactive immune cells and cancer cells.

### SUMMAY

The present inventors have recognised that pre-treatment with PKC inhibitors in a specific time window before treatment with a cytotoxic agent increases the efficacy of treatment. This may be useful, for example in increasing the cell death induced by the cytotoxic agent, reducing side effects and improving treatment outcomes.

In accordance with a first aspect of the invention, there is provided a PKC inhibitor and a cytotoxic agent for use in therapy, wherein the PKC inhibitor reaches a peak concentration in a subject prior to the cytotoxic agent reaching a peak concentration.

Advantageously, the PKC inhibitor increases the sensitivity of the subject to the cytotoxic agent.

In accordance with a second aspect, there is provided a method for increasing the sensitivity of a subject to a cytotoxic agent, the method comprising administering a PKC inhibitor and a cytotoxic agent to the subject, wherein the PKC inhibitor reaches a peak concentration in the subject prior to the cytotoxic agent reaching a peak concentration.

In particular, the inventors have found that the combination of the PKC inhibitor and the cytotoxic agent may be used to treat cancer or an autoimmune disease.

In accordance with a third aspect, there is provided a PKC inhibitor and a cytotoxic agent for use in the treatment of cancer or an autoimmune disease, wherein the PKC inhibitor reaches a peak concentration in a subject prior to the cytotoxic agent reaching a peak concentration.

In accordance with a fourth aspect, there is provided a method treating cancer or an autoimmune disease in a subject, the method comprising administering a PKC inhibitor and a cytotoxic agent to the subject, wherein the PKC inhibitor reaches a peak concentration in the subject prior to the cytotoxic agent reaching a peak concentration. The peak concentration of the PKC inhibitor may be understood to be the maximum concentration that the PKC inhibitor reaches after administration to the subject.

Similarly, the peak concentration of the cytotoxic agent may be understood to be the maximum concentration that the cytotoxic agent reaches after administration to the subject. The peak concentration may be understood to be the maximum concentration of the PKC inhibitor or the cytotoxic agent in the blood, cerebrospinal fluid, a target organ or a tumour. In some embodiments, the peak concentration may be understood to be the maximum concentration of the PKC inhibitor or the cytotoxic agent in the blood.

The PKC inhibitor may be a PKC-β inhibitor. Suitable PKC inhibitors are well-known in the art and include enzastaurin, sotrastaurin, midostaurin (PKC412), MS-553, Gouml 6983, staurosporine, GF 109203X (bisindolylmaleimide I), Go6976, ZIP, LY 333531 hydrochloride (ruboxistaurin), Ro 31-8220 mesylate, Ro 32-0432 hydrochloride, rottlerin, baicalein, quercetin, luteolin, bisindolylmaleimide II, calphostin C, chelerythrine chloride, L-threo dihydrosphingosine (safingol), and melittin.

In some embodiments, the PKC inhibitor may be selected from enzastaurin (3-(1-methylindol-3-yl)-4-[1-[1-(pyridin-2-ylmethyl)piperidin-4-yl]indol-3-yl]pyrrole-2,5-dione; CAS 170364-57-5), sotrastaurin (5-hydroxy-4-(1H-indol-3-yl)-3-[2-(4-methylpiperazin-1-yl)quinazolin-4-yl]-2H-pyrrol-2-one; CAS 425637-18-9), midostaurin (N-((5R,7R,8R,9S)-8-methoxy-9-methyl-16-oxo-6,7,8,9,15,16-hexahydro-5H,14H-17-oxa-4b,9a,15-triaza-5,9-methanodibenzo[b,h]cyclonona[jkl]cyclopenta[e]-as-indacen-7-yl)-N-methylbenzamide; CAS 120685-11-2) and MS-553. In some preferred embodiments, the PKC inhibitor is enzastaurin.

A cytotoxic agent may be understood to be an agent which is toxic to mammalian cells and induces cell death. A cytotoxic agent may directly target cell viability. For example, a cytotoxic agent may target the anti-apoptosis pathway, or be mitotic inhibitors, nucleoside analogues, or DNA-intercalating agents (e.g. anthracyclines). Suitable cytotoxic agents may include alkylating agents, such as bendamustine and chlorambucil; antimetabolites, including purine analogues, such as fludarabine, and cladribine, pyrimidine analogues, such as cytarabine; anti-microtubule agents, such as vincristine; folate antagonists, such as methotrexate; topoisomerase inhibitors; DNA intercalating agents, including anthracyclines, such as doxorubicin and daunorubicin; apoptosis inducers, including BCL-2 inhibitors, such as venetoclax (ABT-199), AZD5991, AMG176, A-1210477 and navitoclax; BTK inhibitors, such as Ibrutinib; P3K inhibitors, such as Idelalisib; gluticosteroids, such as prednisolone and dexamethasone; and cytotoxic antibodies, in particular B cell targeting antibodies, such as rituximab.

In some preferred embodiments, the cytotoxic agent may be selected from fludarabine, cladribine, cytarabine, chlorambucil, venetoclax, navitoclax, AZD5991, AMG176, A-1210477, bendamustine, cyclophosphamide, prednisolone, methotrexate, vincristine, doxorubicin, daunorubicin, and rituximab.

The cytotoxic agent may be a mitosis inhibitor, a nucleoside analogue, an anthracycline, a DNA-intercalating agent, an alkylating agent, an antimetabolite, an anti-microtubule agent, a folate antagonist, a topoisomerase inhibitor, an apoptosis inducer, a BCL-2 inhibitor, a BTK inhibitor, a P3K inhibitor, a glucocorticoid, or a cytotoxic antibody. The cytotoxic agent may be a chemotherapy medication or an immunosuppressant.

The cytotoxic agent may be fludarabine, venetoclax, methotraxate, vincristine, dexamethasone, an anthracycline, bendamustine, idealisib, ibrutinib, methotrexate, cyclophosphamide, a steroid or a monoclonal antibody targeting B cells, or a pharmaceutically acceptable salt or solvate thereof. The anthracycline may be doxorubicin, daunorubicin, epirubicin or idarubicin, or a pharmaceutically acceptable salt or solvate thereof. The steroid may be prednisolone, or a pharmaceutically acceptable salt or solvate thereof. The monoclonal antibody targeting B cells may be rituximab, or a pharmaceutically acceptable salt or solvate thereof. In some embodiments, the cytotoxic agent is fludarabine. In some embodiments, the cytotoxic agent is venetoclax. In some embodiments, the cytotoxic agent is bendamustine.

In some embodiments, the PKC inhibitor and the cytotoxic agent are for use in treating cancer. The cancer may include B-cell malignancy and/or may be a myeloid cancer. The cancer may be selected from the group consisting of lymphoma, leukemia, breast cancer, bile duct cancer, bladder cancer, gastric cancer, lung cancer, prostate cancer, colon cancer and colorectal cancer. The cancer may be a B cell lymphoma. The cancer may be selected from the group consisting of chronic lymphocytic leukemia (CLL), mantle cell lymphoma (MCL), acute lymphoblastic leukemia (B-ALL) and acute myeloid leukemia (AML), follicular lymphoma, diffuse large B cell lymphoma and Burkitt lymphoma. In some embodiments, the cancer is chronic lymphocytic leukemia (CLL). In alternative embodiments, the cancer is acute myeloid leukemia (AML).

The cancer may be a drug resistant cancer.

In some embodiments, the PKC inhibitor and the cytotoxic agent are for use in treating an autoimmune disease. The autoimmune disease may be selected from the group consisting of rheumatoid arthritis, systemic lupus erythematosus, inflammatory bowel disease, multiple sclerosis, diabetes mellitus type 1, celiac disease, Grave's disease, psoriasis, and vasculitis. In some embodiments, the autoimmune diseases is systemic lupus erythematosus (SLE). In alternative embodiments, the autoimmune disease is rheumatoid arthritis (RA).

The PKC inhibitor may reaches a peak concentration in a subject at least 30 minutes prior to the cytotoxic agent reaching a peak concentration, at least 1 hour prior to the cytotoxic agent reaching a peak concentration, at least 2 hours prior to the cytotoxic agent reaching a peak concentration or at least 3 hours prior to the cytotoxic agent reaching a peak concentration. The PKC inhibitor may reaches a peak concentration in a subject less than 12 hours prior to the cytotoxic agent reaching a peak concentration, less than 8 hours prior to the cytotoxic agent reaching a peak concentration, less than 6 hours prior to the cytotoxic agent reaching a peak concentration or less than 5 hours prior to the cytotoxic agent reaching a peak concentration. The PKC inhibitor may reaches a peak concentration in a subject between 30 minutes and 12 hours prior to the cytotoxic agent reaching a peak concentration, between 1 and 8 hours prior to the cytotoxic agent reaching a peak concentration, between 2 and 6 hours prior to the cytotoxic agent reaching a peak concentration or between 3 and 5 hours prior to the cytotoxic agent reaching a peak concentration. Alternatively, the PKC inhibitor may reaches a peak concentration in a subject between 30 minutes and 12 hours prior to the cytotoxic agent reaching a peak concentration, between 45 minutes and 6 hours prior to the cytotoxic agent reaching a peak concentration, between 1 and 5 hours prior to the cytotoxic agent reaching a peak concentration or between 1 and 4 hours prior to the cytotoxic agent reaching a peak concentration.

Medicaments comprising the PKC inhibitor and/or the cytotoxic agent described herein may be used in a number of ways. Compositions comprising the PKC inhibitor and/or the cytotoxic agent of the invention may be administered by inhalation (e.g. intranasally). Compositions may also be formulated for topical use. For instance, creams or ointments may be applied to the skin.

The PKC inhibitor and/or the cytotoxic agent and compositions according to the invention may be administered to a subject by injection into the blood stream or directly into a site requiring treatment, for example into a cancerous tumour or into the blood stream adjacent thereto. Injections may be intravenous (bolus or infusion) or subcutaneous (bolus or infusion), intradermal (bolus or infusion) or intramuscular (bolus or infusion).

The PKC inhibitor and/or the cytotoxic agent may be administered orally. Accordingly, the PKC inhibitor and/or the cytotoxic agent may be contained within a composition that may, for example, be ingested orally in the form of a tablet, capsule or liquid.

It will be appreciated that the amount of the PKC inhibitor and/or the cytotoxic agent that is required is determined by its biological activity and bioavailability, which in turn depends on the mode of administration, the physiochemical properties of the PKC inhibitor and/or the cytotoxic agent, and whether it is being used as a monotherapy, or in a combined therapy. The frequency of administration will also be influenced by the half-life of the PKC inhibitor and/or the cytotoxic agent within the subject being treated. Optimal dosages to be administered may be determined by those skilled in the art, and will vary with the particular PKC inhibitor and/or the cytotoxic agent in use, the strength of the pharmaceutical composition, the mode of administration, and the advancement of the disease. Additional factors depending on the particular subject being treated will result in a need to adjust dosages, including subject age, weight, sex, diet, and time of administration.

The inhibitor may be administered before, during or after onset of the disease to be treated. Daily doses may be given as a single administration. Alternatively, the PKC inhibitor and/or the cytotoxic agent may be given two or more times during a day, and most preferably twice a day.

Generally, a daily dose of between 0.01 μg/kg of body weight and 500 mg/kg of body weight of the PKC inhibitor and/or the cytotoxic agent according to the invention may be used. More preferably, the daily dose is between 0.01 mg/kg of body weight and 400 mg/kg of body weight, more preferably between 0.1 mg/kg and 200 mg/kg body weight, and most preferably between approximately 1 mg/kg and 100 mg/kg body weight.

A patient receiving treatment may take a first dose upon waking and then a second dose in the evening (if on a two dose regime) or at 3- or 4-hourly intervals thereafter. Alternatively, a slow release device may be used to provide optimal doses of the inhibitor according to the invention to a patient without the need to administer repeated doses.

Known procedures, such as those conventionally employed by the pharmaceutical industry (e.g. in vivo experimentation, clinical trials, etc.), may be used to form specific formulations comprising the PKC inhibitor and/or the cytotoxic agent according to the invention and precise therapeutic regimes (such as daily doses of the inhibitor and the frequency of administration). The inventors believe that they are the first to describe a pharmaceutical composition based on the use of a PKC inhibitor and a cytotoxic agent.

Accordingly, in a fifth aspect, there is provided a pharmaceutical composition, the composition comprising a PKC inhibitor and a cytotoxic agent, or a pharmaceutically acceptable salt or solvate thereof, and a pharmaceutically acceptable vehicle.

Preferably, the composition is configured to ensure that after administration thereof, the PKC inhibitor reaches a peak concentration in a subject prior to the cytotoxic agent reaching a peak concentration. This may be achieved using a delayed release formulation. Alternatively, depending upon the PKC inhibitor and the cytotoxic agent such a formulation may not be required. For instance, PKC inhibitors tend to reach a peak concentration about three hours after administration. Conversely, BCL-2 inhibitors tend to reach peak concentration about 8 hours after administration. Accordingly, if a composition comprising a PKC inhibitors and a BCL-2 inhibitor was administered to a patient, the PKC inhibitor would reaches a peak concentration in the subject about 5 hours prior to the BCL-2 inhibitor reaching a peak concentration.

The invention also provides, in a sixth aspect, a process for making the composition according to the fifth aspect, the process comprising contacting a therapeutically effective amount of a PKC inhibitor, or a pharmaceutically acceptable salt or solvate thereof, a cytotoxic agent, or a pharmaceutically acceptable salt or solvate thereof, and a pharmaceutically acceptable vehicle.

A “subject” may be a vertebrate, mammal, or domestic animal. Hence, the PCK inhibitor and the cytotoxic agent may be used to treat any mammal, for example livestock (e.g. a horse), pets, or may be used in other veterinary applications. Most preferably, however, the subject is a human being.

A “therapeutically effective amount” of the PCK inhibitor and the cytotoxic agent is any amount which, when administered to a subject, is the amount of drug that is needed to treat the cancer or autoimmune disease.

For example, the therapeutically effective amount of the PCK inhibitor and the cytotoxic agent used may be from about 0.01 mg to about 800 mg, and preferably from about 0.01 mg to about 500 mg. It is preferred that the amount of the PCK inhibitor and the cytotoxic agent is an amount from about 0.1 mg to about 250 mg, and most preferably from about 0.1 mg to about 20 mg.

A “pharmaceutically acceptable vehicle” as referred to herein, is any known compound or combination of known compounds that are known to those skilled in the art to be useful in formulating pharmaceutical compositions.

In one embodiment, the pharmaceutically acceptable vehicle may be a solid, and the composition may be in the form of a powder or tablet. A solid pharmaceutically acceptable vehicle may include one or more substances which may also act as flavouring agents, lubricants, solubilisers, suspending agents, dyes, fillers, glidants, compression aids, inert binders, sweeteners, preservatives, dyes, coatings, or tablet-disintegrating agents. The vehicle may also be an encapsulating material. In powders, the vehicle is a finely divided solid that is in admixture with the finely divided active agents (i.e. the inhibitor) according to the invention. In tablets, the inhibitor may be mixed with a vehicle having the necessary compression properties in suitable proportions and compacted in the shape and size desired. The powders and tablets preferably contain up to 99% of the inhibitor. Suitable solid vehicles include, for example calcium phosphate, magnesium stearate, talc, sugars, lactose, dextrin, starch, gelatin, cellulose, polyvinylpyrrolidine, low melting waxes and ion exchange resins. In another embodiment, the pharmaceutical vehicle may be a gel and the composition may be in the form of a cream or the like.

However, the pharmaceutical vehicle may be a liquid, and the pharmaceutical composition is in the form of a solution. Liquid vehicles are used in preparing solutions, suspensions, emulsions, syrups, elixirs and pressurized compositions. The inhibitor according to the invention may be dissolved or suspended in a pharmaceutically acceptable liquid vehicle such as water, an organic solvent, a mixture of both or pharmaceutically acceptable oils or fats. The liquid vehicle can contain other suitable pharmaceutical additives such as solubilisers, emulsifiers, buffers, preservatives, sweeteners, flavouring agents, suspending agents, thickening agents, colours, viscosity regulators, stabilizers or osmo-regulators. Suitable examples of liquid vehicles for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the vehicle can also be an oily ester such as ethyl oleate and isopropyl myristate. Sterile liquid vehicles are useful in sterile liquid form compositions for parenteral administration. The liquid vehicle for pressurized compositions can be a halogenated hydrocarbon or other pharmaceutically acceptable propellant.

Liquid pharmaceutical compositions, which are sterile solutions or suspensions, can be utilized by, for example, intramuscular, intrathecal, epidural, intraperitoneal, intravenous and particularly subcutaneous injection. The inhibitor may be prepared as a sterile solid composition that may be dissolved or suspended at the time of administration using sterile water, saline, or other appropriate sterile injectable medium.

The PCK inhibitor and/or the cytotoxic agent of the invention may be administered in the form of a sterile solution or suspension containing other solutes or suspending agents (for example, enough saline or glucose to make the solution isotonic), bile salts, acacia, gelatin, sorbitan monoleate, polysorbate 80 (oleate esters of sorbitol and its anhydrides copolymerized with ethylene oxide) and the like. The PCK inhibitor and/or the cytotoxic agent used according to the invention can also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

A seventh aspect provides a method for increasing the sensitivity of a subject to a cytotoxic agent comprising administering a PKC inhibitor in combination with a cytotoxic agent to the subject, wherein the PKC inhibitor is administered to the subject 2-6 hours prior to administration of the cytotoxic agent.

The PKC inhibitor may increase the sensitivity of disease cells, such as auto-reactive immune cells and cancer cells, including malignant B cells, of the subject to the cytotoxic agent.

An eighth aspect of the invention provides a method of treating cancer in a subject comprising administering a PKC inhibitor in combination with a cytotoxic agent to the subject, wherein the PKC inhibitor is administered to the subject 2-6 hours prior to administration of the cytotoxic agent.

A ninth aspect of the invention provides a method of treating an autoimmune disease in a subject comprising administering a PKC inhibitor in combination with a cytotoxic agent to the subject, wherein the PKC inhibitor is administered to the subject 2-6 hours prior to administration of the cytotoxic agent.

A tenth aspect of the invention provides a PKC inhibitor for use in a method according to any one of the seventh, eighth or ninth aspects.

An eleventh aspect of the invention provides the use of a PKC inhibitor in the manufacture of a medicament for use in for use in a method according to any one of the seventh, eighth or ninth aspects.

A twelfth aspect of the invention provides a cytotoxic agent for use in a method according to any one of the seventh, eighth or ninth aspects.

A thirteenth aspect of the invention provides the use of a cytotoxic agent in the manufacture of a medicament for use in a method according to any one of the seventh, eighth or ninth aspects.

A fourteenth aspect of the invention provides a combination of a PKC inhibitor and a cytotoxic agent for use in a method according to any one of the seventh, eighth or ninth aspects.

A fifteenth aspect of the invention provides the use of a combination of a PKC inhibitor and a cytotoxic agent in the manufacture of a medicament for use in a method according to any one of the seventh, eighth or ninth aspects.

Preferred PKC inhibitors for use in the first to the ninth aspects include enzastaurin, sotrastaurin and midostaurin, preferably enzastaurin.

Preferred cytotoxic agents for use in the first to the ninth aspects include fludarabine, methotraxate, vincristine, doxorubicin and other anthracyclines, bendamustine, cyclophosphamide, steroids, such as prednisolone, and monoclonal antibodies targeting B cells, such as rituximab.

Cancers treated in the in the second and the fourth to the ninth aspects may include B-cell malignancies and myeloid cancers.

Autoimmune diseases treated in the third to the ninth aspects may include systemic lupus erythematosus (SLE) and rheumatoid arthritis (RA).

In another aspect, this invention relates to the finding that administration of a PKC inhibitor within a specific time window before the administration of a cytotoxic agent significantly enhances chemosensitisation, increasing the efficacy of the cytotoxic agent. This effect is not observed when the PKC inhibitor is administered outside the time window. The PKC inhibitor may, for example, sensitize disease cells, such as tumor cells, to the cytotoxic agent and increase cell death compared to treatment with the cytotoxic agent alone. The PKC inhibitor may, in some embodiments, antagonize environment-mediated resistance to the cytotoxic agent. These findings may be useful in improving patient outcomes by enhancing the effectiveness of therapies, for example, cancer therapies or autoimmune disease therapies, by mitigating side effects and/or by reducing the effective dose of cytotoxic agents.

The PKC inhibitor may be administered to the subject 2-6 hours before the administration of the cytotoxic agent, for example 2-5 hours, 2-4 hours, 2-3 hours, 3-6 hours, 3-5 hours, 3-4 hours, 4-6 hours, 4-5 hours, or 5-6 hours. In some preferred embodiments the PKC inhibitor may be administered to the subject 3-6 hours before the administration of the cytotoxic agent. Administration of the PKC inhibitor inside this time window is shown to increase the efficacy and/or cytotoxic effect of the cytotoxic agent, whereas administration outside this specific time window has no such effect.

In some embodiments, the optimal time-point for administration of the PKC inhibitor to the subject within the 2-6 hour time window before the administration of the cytotoxic agent may vary depending on the specific dosage and specific PKC inhibitor and cytotoxic agent used.

Protein kinase C (PKC; (EC 2.7.11.13) is a serine/threonine protein kinase family of enzymes that transduce signals and regulate other proteins through phosphorylation. The family consists of several isozymes, including PKC-α, PKC-β, PKC-γ, PKC-η, PKC-ε, PKC-δ, PKC-θ, PKC-ι, PKC-ξ, PRK1 and PRK2. PKC isozymes play major roles in the control of signalling pathways associated with proliferation, migration, invasion, tumorigenesis, and metastasis. More recently, PKC-β transduction of anti-apoptotic signals has been linked to environment-mediated drug resistance. A PKC inhibitor may be selective for one or more PKC isotypes, preferably PKC-β.

Preferred PKC inhibitors for use as described herein inhibit PKC-β (i.e. the PKC inhibitor is preferably a PKC-β inhibitor).

A PKC inhibitor is an agent which inhibits the activity or reduces/inhibits the expression of PKC.

Suitable agents for inhibiting the activity or reducing/inhibiting the expression of PKC include antibodies and other immunoglobulin molecules, aptamers, suppressor nucleic acids, and small chemical molecules, for example non-polymeric organic compounds having a molecular weight of 900 Daltons or less.

Suitable PKC inhibitors are well-known in the art and include enzastaurin, sotrastaurin, midostaurin (PKC412), MS-553, Gouml 6983, staurosporine, GF 109203X (bisindolylmaleimide I), Go6976, ZIP, LY 333531 hydrochloride (ruboxistaurin), Ro 31-8220 mesylate, Ro 32-0432 hydrochloride, rottlerin, baicalein, quercetin, luteolin, bisindolylmaleimide II, calphostin C, chelerythrine chloride, L-threo dihydrosphingosine (safingol), and melittin.

In some embodiments, the PKC inhibitor may be selected from enzastaurin (3-(1-methylindol-3-yl)-4-[1-[1-(pyridin-2-ylmethyl)piperidin-4-yl]indol-3-yl]pyrrole-2,5-dione; CAS 170364-57-5), sotrastaurin (5-hydroxy-4-(1H-indol-3-yl)-3-[2-(4-methylpiperazin-1-yl)quinazolin-4-yl]-2H-pyrrol-2-one; CAS 425637-18-9), midostaurin (N-((5R,7R,8R,9S)-8-methoxy-9-methyl-16-oxo-6,7,8,9,15,16-hexahydro-5H,14H-17-oxa-4b,9a,15-triaza-5,9-methanodibenzo[b,h]cyclonona[jkl]cyclopenta[e]-as-indacen-7-yl)-N-methylbenzamide; CAS 120685-11-2) and MS-553. In some preferred embodiments, the PKC inhibitor is enzastaurin.

The PKC inhibitor is administered to the subject in combination with a cytotoxic agent in the methods described herein.

A cytotoxic agent is an agent which is toxic to mammalian cells and induces cell death. A cytotoxic agent may directly target cell viability. For example, a cytotoxic agent may target the anti-apoptosis pathway, or be mitotic inhibitors, nucleoside analogues, or DNA-intercalating agents (e.g. anthracyclines). Suitable cytotoxic agents may include alkylating agents, such as bendamustine and chlorambucil; antimetabolites, including purine analogues, such as fludarabine, and cladribine, pyrimidine analogues, such as cytarabine; anti-microtubule agents, such as vincristine; folate antagonists, such as methotrexate; topoisomerase inhibitors; DNA intercalating agents, including anthracyclines, such as doxorubicin and daunorubicin; apoptosis inducers, including BCL-2 inhibitors, such as venetoclax (ABT-199), AZD5991, AMG176, A-1210477 and navitoclax; BTK inhibitors, such as Ibrutinib; P3K inhibitors, such as Idelalisib; gluticosteroids, such as prednisolone and dexamethasone; and cytotoxic antibodies, in particular B cell targeting antibodies, such as rituximab.

In some preferred embodiments, the cytotoxic agent may be selected from fludarabine, cladribine, cytarabine, chlorambucil, venetoclax, navitoclax, AZD5991, AMG176, A-1210477, bendamustine, cyclophosphamide, prednisolone, methotrexate, vincristine, doxorubicin, daunorubicin, and rituximab.

While it is possible for a cytotoxic agent or a PKC inhibitor to be administered to the individual alone, it is preferable to present the cytotoxic agent or PKC inhibitor in a pharmaceutical composition or formulation.

A pharmaceutical composition may comprise, in addition to the active compound(s), one or more pharmaceutically acceptable carriers, adjuvants, excipients, diluents, fillers, buffers, stabilisers, preservatives, lubricants, or other materials well-known to those skilled in the art. Such materials should be non-toxic and should not interfere with the efficacy of the active compound. The precise nature of the carrier or other material will depend on the route of administration, which may be by bolus, infusion, injection or any other suitable route, as discussed below. Suitable materials will be sterile and pyrogen free, with a suitable isotonicity and stability. Examples include sterile saline (e.g. 0.9% NaCl), water, dextrose, glycerol, ethanol or the like or combinations thereof. The composition may further contain auxiliary substances such as wetting agents, emulsifying agents, pH buffering agents or the like.

Suitable carriers, excipients, etc. can be found in standard pharmaceutical texts, for example, Remington's Pharmaceutical Sciences, 18th edition, Mack Publishing Company, Easton, Pa., 1990.

The term “pharmaceutically acceptable” as used herein pertains to compounds, materials, compositions, and/or dosage forms which are, within the scope of sound medical judgement, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

The formulations may conveniently be presented in unit dosage form and may be prepared by any methods well-known in the art of pharmacy. Such methods include the step of bringing into association the active compound with the carrier which constitutes one or more accessory ingredients. In general, the formulations are prepared by uniformly and intimately bringing into association the active compound with liquid carriers or finely divided solid carriers or both, and then if necessary shaping the product.

Formulations may be in the form of liquids, solutions, suspensions, emulsions, elixirs, syrups, tablets, lozenges, granules, powders, capsules, cachets, pills, ampoules, suppositories, pessaries, ointments, gels, pastes, creams, sprays, mists, foams, lotions, oils, boluses, electuaries, or aerosols.

The active compounds or pharmaceutical compositions comprising the active compounds may be administered to a subject by any convenient route of administration, whether systemically/peripherally or at the site of desired action, including but not limited to, oral (e.g. by ingestion); and parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot, for example, subcutaneously or intramuscularly. Usually administration will be by the oral route, although other routes such as intraperitoneal, subcutaneous, transdermal, intravenous, nasal, intramuscular or other convenient routes are not excluded.

The pharmaceutical compositions comprising the active compounds may be formulated in a dosage unit formulation that is appropriate for the intended route of administration.

Formulations suitable for oral administration (e.g. by ingestion) may be presented as discrete units such as capsules, cachets or tablets, each containing a predetermined amount of the active compound; as a powder or granules; as a solution or suspension in an aqueous or non-aqueous liquid; or as an oil-in-water liquid emulsion or a water-in-oil liquid emulsion; as a bolus; as an electuary; or as a paste.

A tablet may be made by conventional means, e.g., compression or moulding, optionally with one or more accessory ingredients. Compressed tablets may be prepared by compressing in a suitable machine the active compound in a free-flowing form such as a powder or granules, optionally mixed with one or more binders (e.g. povidone, gelatin, acacia, sorbitol, tragacanth, hydroxypropylmethyl cellulose); fillers or diluents (e.g. lactose, microcrystalline cellulose, calcium hydrogen phosphate); lubricants (e.g. magnesium stearate, talc, silica); disintegrants (e.g. sodium starch glycolate, cross-linked povidone, cross-linked sodium carboxymethyl cellulose); surface-active or dispersing or wetting agents (e.g. sodium lauryl sulfate); and preservatives (e.g. methyl p-hydroxybenzoate, propyl p-hydroxybenzoate, ascorbic acid). Moulded tablets may be made by moulding in a suitable machine a mixture of the powdered compound moistened with an inert liquid diluent. The tablets may optionally be coated or scored and may be formulated so as to provide slow or controlled release of the active compound therein using, for example, hydroxypropylmethyl cellulose in varying proportions to provide the desired release profile. Tablets may optionally be provided with an enteric coating, to provide release in parts of the gut other than the stomach.

Formulations suitable for parenteral administration (e.g. by injection, including cutaneous, subcutaneous, intramuscular, intravenous and intradermal), include aqueous and non-aqueous isotonic, pyrogen-free, sterile injection solutions which may contain anti-oxidants, buffers, preservatives, stabilisers, bacteriostats, and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. Examples of suitable isotonic vehicles for use in such formulations include Sodium Chloride Injection, Ringer's Solution, or Lactated Ringer's Injection. Typically, the concentration of the active compound in the solution is from about 1 ng/ml to about 10 μg/ml, for example, from about 10 ng/ml to about 1 μg/ml. The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules, and tablets. Formulations may be in the form of liposomes or other microparticulate systems which are designed to target the active compound to blood components or one or more organs.

An individual or subject suitable for treatment as described herein may have a disease condition. For example, one or more cells of the individual may be disease cells.

In some embodiments, the individual may have cancer. For example, one or more cells of the individual may be cancer cells. Cancer includes any unwanted cell proliferation (or any disease manifesting itself by unwanted cell proliferation), neoplasm or tumour or increased risk of or predisposition to the unwanted cell proliferation, neoplasm or tumour. The cancer may be benign or malignant and may be primary or secondary (metastatic). Cancer suitable for treatment as described herein may be any type of solid or non-solid cancer or malignant lymphoma and especially leukaemia, sarcomas, skin cancer, bladder cancer, blood cancer, breast cancer, uterine cancer, ovarian cancer, prostate cancer, lung cancer, colorectal cancer, cervical cancer, liver cancer, head and neck cancer, oesophageal cancer, pancreatic cancer, renal cancer, stomach cancer and cerebral cancer. Cancers may be familial or sporadic.

In preferred embodiments, the cancer is a blood cancer. Blood cancer may include a B cell malignancy i.e. a cancer affecting B cells. For example, the cancer cells may be malignant B cells. B cell malignancies may include lymphomas, such as non-Hodgkin's lymphoma (NHL), Hodgkin's lymphoma (HL), Burkitt's lymphoma, diffuse large B-cell lymphoma, mantle cell lymphoma (MCL), and follicular lymphoma, and leukaemia, such as chronic lymphocytic leukemia (CLL), acute lymphoblastic leukemia (B-ALL) and acute myeloid leukemia (AML). In some preferred embodiments, the cancer is chronic lymphoid leukemia (CLL). In other preferred embodiments the cancer is acute myeloid leukemia (AML).

In some embodiments, the cancer may be resistant to the cytotoxic agent in the absence of the PKC inhibitor and/or resistant to the PKC inhibitor in the absence of the cytotoxic agent. For example, the cancer may display environment-mediated resistance to the cytotoxic agent. A PKC inhibitor administered in the defined time window described herein may antagonise environment-mediated drug resistance and sensitise tumour cells to the cytotoxic agent, leading to enhanced cytotoxicity and efficacy.

In some embodiments, the individual may have minimal residual disease (MRD) after an initial cancer treatment.

In other embodiments, the individual may have an autoimmune disease. For example, one or more cells of the individual may be autoreactive immune cells.

Autoimmune disease is a disease in which the immune system of a subject produces antibodies that attack the subject's normal body tissues. Autoimmune disease may be an autoimmune disease of the nervous, gastrointestinal, blood and blood vessel, skin, endocrine, and/or musculoskeletal systems. Autoimmune diseases suitable for treatment as described herein include rheumatoid arthritis, systemic lupus erythematosus, inflammatory bowel disease, multiple sclerosis, diabetes mellitus type 1, celiac disease, Grave's disease and psoriasis. In some preferred embodiments the autoimmune disease is systemic lupus erythematosus (SLE). In other preferred embodiments, the autoimmune disease is rheumatoid arthritis (RA). A PKC inhibitor administered in the defined time window described herein may antagonise environment-mediated resistance and sensitise autoreactive immune cells to the cytotoxic agent, leading to enhanced cytotoxicity and efficacy.

An individual suitable for treatment as described above may be a mammal, such as a rodent (e.g. a guinea pig, a hamster, a rat, a mouse), murine (e.g. a mouse), canine (e.g. a dog), feline (e.g. a cat), equine (e.g. a horse), a primate, simian (e.g. a monkey or ape), a monkey (e.g. marmoset, baboon), an ape (e.g. gorilla, chimpanzee, orang-utan, gibbon), or a human.

In some preferred embodiments, the individual is a human. In other preferred embodiments, non-human mammals, especially mammals that are conventionally used as models for demonstrating therapeutic efficacy in humans (e.g. murine, primate, porcine, canine, or leporid) may be employed.

An individual with a disease, such as cancer or an autoimmune disease may display at least one identifiable sign, symptom, or laboratory finding that is sufficient to make a diagnosis of the disease in accordance with clinical standards known in the art.

Examples of such clinical standards can be found in textbooks of medicine such as Harrison's Principles of Internal Medicine, 15th Ed., Fauci AS et al., eds., McGraw-Hill, New York, 2001. In some instances, a diagnosis of a disease, such as cancer or an autoimmune disease in an individual may include identification of a particular cell type (e.g. a cancer cell) in a sample of a body fluid or tissue obtained from the individual. In some embodiments, the individual may have been previously identified or diagnosed with a disease, such as cancer or an autoimmune disease, or a method of the invention may comprise identifying or diagnosing the disease in the individual for example by determining the presence of an identifiable sign, symptom, or laboratory finding indicative of the disease in the individual.

Treatment may be any treatment and therapy, whether of a human or an animal (e.g. in veterinary applications), in which some desired therapeutic effect is achieved, for example, the inhibition or delay of the progress of the condition, and includes a reduction in the rate of progress, a halt in the rate of progress, amelioration of the condition, cure or remission (whether partial or total) of the condition, preventing, delaying, abating or arresting one or more symptoms and/or signs of the condition or prolonging survival of a subject or patient beyond that expected in the absence of treatment.

Treatment of a cancer may include inhibiting cancer growth, including complete cancer remission, and/or inhibiting cancer metastasis. Cancer growth generally refers to any one of a number of indices that indicate change within the cancer to a more developed form. Thus, indices for measuring an inhibition of cancer growth include a decrease in cancer cell survival, a decrease in tumor volume or morphology (for example, as determined using computed tomographic (CT), sonography, or other imaging method), a delayed tumor growth, a destruction of tumor vasculature, improved performance in delayed hypersensitivity skin test, an increase in the activity of cytolytic immune cells, and a decrease in levels of tumor-specific antigens.

The PKC inhibitor and cytotoxic agent may be administered as described herein in therapeutically-effective amounts. The term “therapeutically-effective amount” as used herein, pertains to that amount of an active compound, or a combination, material, composition or dosage form comprising an active compound, which is effective for producing some desired therapeutic effect, commensurate with a reasonable benefit/risk ratio.

The appropriate dosage of PKC inhibitors and cytotoxic agents may vary from individual to individual. Determining the optimal dosage will generally involve the balancing of the level of therapeutic benefit against any risk or deleterious side effects of the administration. The selected dosage level will depend on a variety of factors including, but not limited to, the route of administration, the time of administration, the rate of excretion of the active compound, other drugs, compounds, and/or materials used in combination, and the age, sex, weight, condition, general health, and prior medical history of the individual. The amount of active compounds and route of administration will ultimately be at the discretion of the physician, although generally the dosage will be to achieve therapeutic plasma concentrations of the active compound without causing substantial harmful or deleterious side-effects.

In general, a suitable dose of the active compound is in the range of about 100 μg to about 400 mg per kilogram body weight of the subject per day, preferably 200 μg to about 200 mg per kilogram body weight of the subject per day. Where the active compound is a salt, an ester, prodrug, or the like, the amount administered is calculated on the basis of the parent compound and so the actual weight to be used is increased proportionately.

Methods of determining the most effective means and dosage of administration are well known in the art and will vary with the formulation used for therapy, the purpose of the therapy, the target cell being treated, and the subject being treated. Single or multiple administrations can be carried out with the dose level and pattern being selected by the physician.

In some embodiments the administration of the PKC inhibitor enhances the cytotoxic effect of the cytotoxic agent. The cytotoxic effect is “enhanced” when administration of the cytotoxic agent following the administration of the PKC inhibitor results in a greater therapeutic effect (i.e. greater cell death) than when the cytotoxic agent administered alone.

In some embodiments, the administration of the PKC inhibitor may enhance immunosuppression by the cytotoxic agent. The immunosuppression is “enhanced” when administration of the cytotoxic agent following the administration of the PKC inhibitor results in a greater therapeutic effect (i.e. for example increased death of autoreactive T or B cells) than when the cytotoxic agent administered alone.

Other aspects and embodiments of the invention provide the aspects and embodiments described above with the term “comprising” replaced by the term “consisting of” and the aspects and embodiments described above with the term “comprising” replaced by the term “consisting essentially of”.

It is to be understood that the application discloses all combinations of any of the above aspects and embodiments described above with each other, unless the context demands otherwise. Similarly, the application discloses all combinations of the preferred and/or optional features either singly or together with any of the other aspects, unless the context demands otherwise.

Modifications of the above embodiments, further embodiments and modifications thereof will be apparent to the skilled person on reading this disclosure, and as such, these are within the scope of the present invention.

All documents and sequence database entries mentioned in this specification are incorporated herein by reference in their entirety for all purposes.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

All features described herein (including any accompanying claims, abstract and drawings), and/or all of the steps of any method or process so disclosed, may be combined with any of the above aspects in any combination, except combinations where at least some of such features and/or steps are mutually exclusive.

### BRIEF DESCRIPTION OF FIGURES

For a better understanding of the invention, and to show how embodiments of the same may be carried into effect, reference will now be made, by way of example, to the accompanying Figures, in which:

FIG. 1 shows the effect of Enzastaurin dosed for 1 hour at indicated hours pre-Fludarabine treatment on the efficacy of cytotoxic agent Fludarabine; FIG. 1A shows the dosing regimen for each of the experimental conditions; and FIG. 1B shows the percentage of live cancer cells after treatment;

FIG. 2 shows the effect of Enzastaurin dosed for 1 hour at indicated hours pre-Ventoclax treatment on the efficacy of cytotoxic agent Venetoclax; FIG. 2A shows the dosing regimen for each of the experimental conditions; and FIG. 2B shows the percentage of live cancer cells after treatment;

FIG. 3 shows the effect of Enzastaurin dosed for the indicated hours including 24 hour Ventoclax treatment on the efficacy of cytotoxic agent Venetoclax; FIG. 3A shows the dosing regimen for each of the experimental conditions; FIG. 3B shows the percentage of live cancer cells after treatment;

FIG. 4 shows IC₅₀ graphs of human CLL cells after 72 hours mono-culture, PKC-β WT co-culture or PKC-β KO co-culture, respectively, in the presence of Venetoclax, Bendamustine, Fludarabine, Ibrutinib or Idelalisib treatment administered 24 hours post-seeding of CLL (n=5 patients per culture condition). CLL viabilities were normalized to respective DMSO controls. Statistical significance between PKC-β WT and PKC-β KO are shown, using paired, two-tail Student t-tests;

FIG. 5 shows synergism as calculated using Compusyn Software (CRUK), within the Bliss model, for Venetoclax combined with Enzastaurin, Sotrastaurin, or Midostaurin, respectively (n=6). Heatmaps reflect assessment values noted for respective compound combinations, with error (*) indicated below. A scale of 50 to −50, is applied to values, with 50 representing maximal synergism and −50 being maximal antagonism. Non-significant values are by default coloured as neither synergistic nor antagonistic;

FIG. 6 shows PKC-β expression in mesenchymal stromal cells (MSCs) is essential for normal B1 cell development. FIG. 6A shows an experimental schematic to assess the functional consequence of adoptive transfer of CD45⁺ selected PKC-β WT bone marrow cells (BM) or KO BM, respectively, into lethally-irradiated (10 Gy) PKC-β WT or KO recipients. FIG. 6B shows an assessment of chimerism in mice with mismatched CD45 isotypes. Percentages of respective donor CD45 isotype are shown ±SEM. Genotype of donor BM is italicized; WT:WT (n=3), KO:WT (n=4), and WT:KO (n=4). Abbreviations for tissues as follows: PB, peripheral blood; PC, peritoneal cavity; SPC, splenocytes. FIG. 6C shows non-irradiated WT control (n=3), WT:KO (n=7), and 4 respective individuals of WT:WT, KO:WT, and KO:KO respectively, assessed for peritoneal CD19+CD5+IgM+ cells with the label of donor cells italicized;

FIG. 7 shows the effect of Enzastaurin dosed for 1 hour at indicated hours pre-Bendamustine treatment on the efficacy of cytotoxic agent Bendamustine. The figure shows the percentage of live cancer cells after treatment;

FIG. 8 shows the effect of Enzastaurin dosed for the indicated hours pre-Bendamustine treatment on the efficacy of cytotoxic agent Bendamustine. The figure shows the percentage of live cancer cells after treatment;

FIG. 9 shows the effect of Midostaurin dosed for the indicated hours pre-Bendamustine treatment on the efficacy of cytotoxic agent Bendamustine. The figure shows the percentage of live cancer cells after treatment; and

FIG. 10 shows the effect of Ruboxistaurin dosed for 1 hour at indicated hours pre-Ventoclax treatment on the observed efficacy of the cytotoxic agent Venetoclax; FIG. 10A shows the dosing regimen for each of the experimental conditions; and FIG. 10B shows the percentage of live cancer cells after treatment.

Experimental

### Example 1—Pre-Treatment with Enzastaurin Increases the Efficacy of Fludarabine and Venetoclax

1-Hour Exposure to Enzastaurin

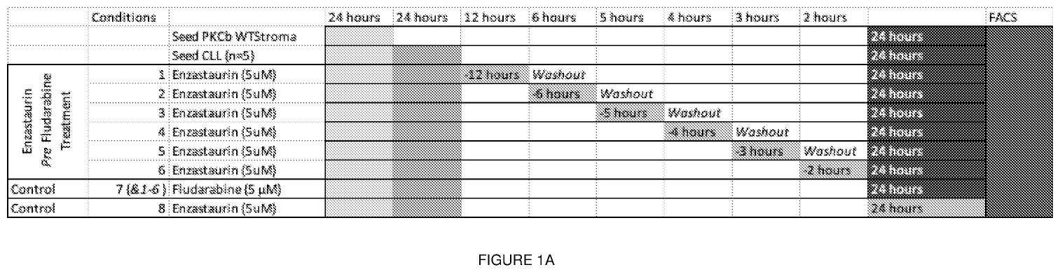

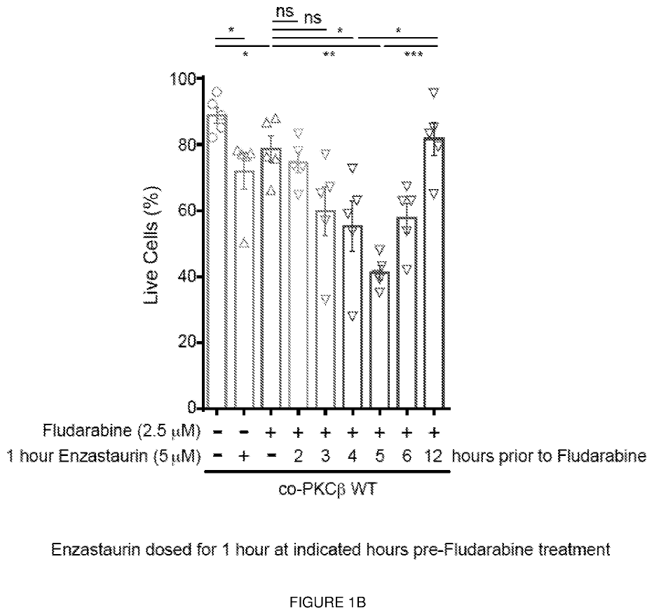

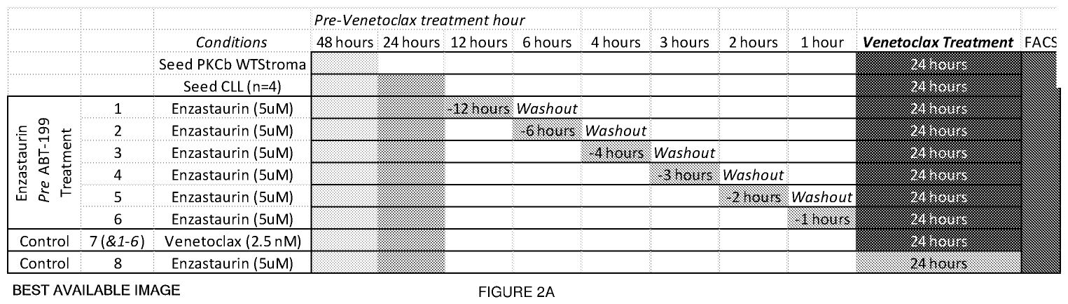

Approximately 20,000 wild-type bone marrow derived stroma cells were seeded into tissue culture wells and subsequently co-cultured with approximately 200,000 CLL patient cells. 24 hours thereafter, co-cultures were treated with 5 μM Enzastaurin for 1 hour at the indicated time-points prior to treatment with Fludarabine or Venetoclax (FIGS. 1A and 2A). Equivalent PKC-β-inhibitor exposure was maintained through removal and subsequent washout of Enzastaurin-treated co-cultures. Co-cultures were centrifuged at 500 g for 5 minutes, prior to removal of Enzastaurin containing media. Washout with cell culture media, was followed by an additional centrifugation at 500 g for 5 minutes, and the replacement of washout media with fresh media. Respective co-cultures were subsequently treated with either Fludarabine (2.5 μM) or Venetoclax (2.5 nM) for 24 hours. Cells were subsequently evaluated for viability using Annexin-V-FITC and DAPI, or 4′,6-diamidino-2-phenylindole, staining on a flow cytometer (FIGS. 1B and 2B).

Continuous Exposure to Enzastaurin

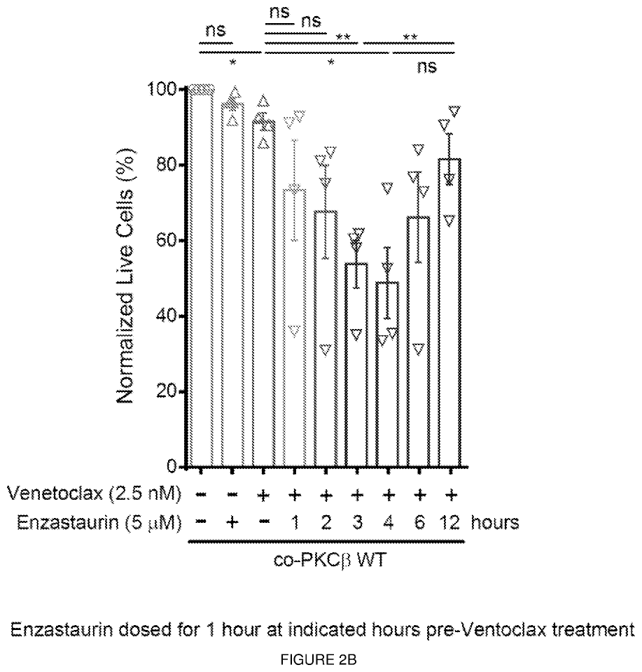

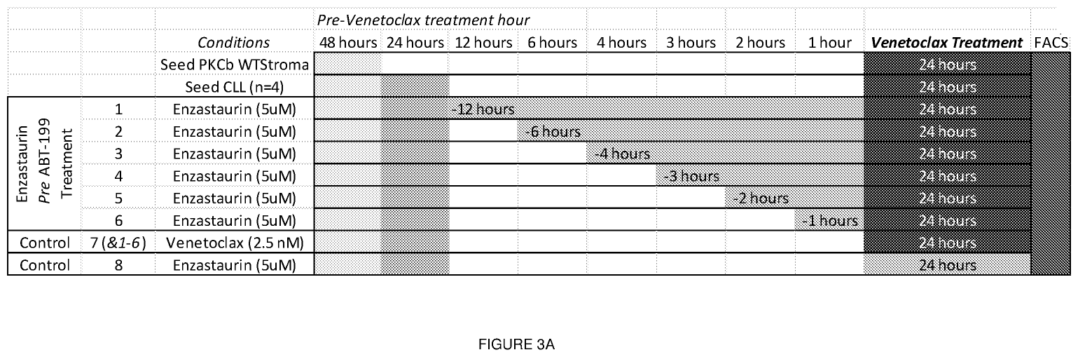

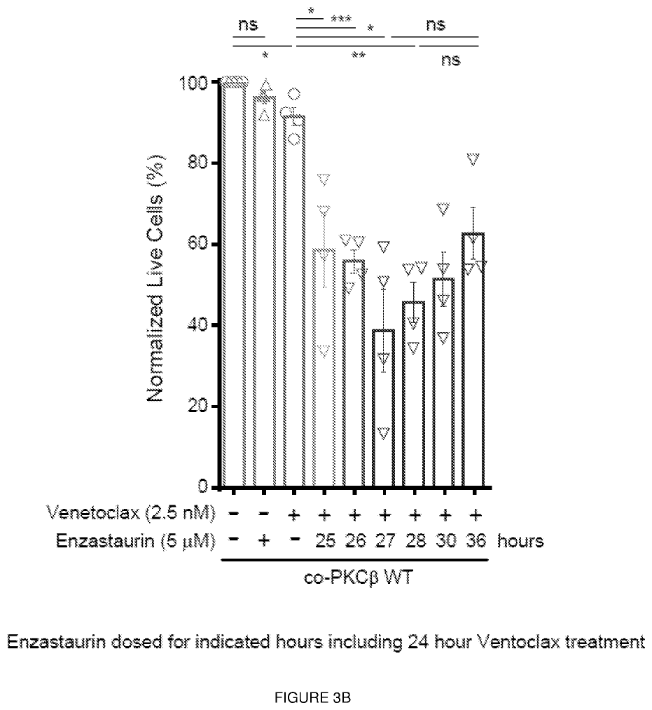

Approximately 20,000 wild-type bone marrow derived stroma cells were seeded into tissue culture wells and subsequently co-cultured with approximately 200,000 CLL patient cells. 24 hours thereafter, co-cultures were treated with 5 μM Enzastaurin at the indicated time-points prior to treatment with Venetoclax (FIG. 3A). Co-cultures were subsequently treated with Venetoclax (2.5 nM) for 24 hours. Cells were subsequently evaluated for viability using Annexin-V-FITC and DAPI staining on a flow cytometer (FIG. 3B).

Results

Pre-treatment with equivalent Enzastaurin exposure showed increased efficacy for both cytotoxic agents at pre-dosing time points of 2-6 hours with the largest effect being observed at 3-6 hours (FIGS. 1B and 2B). Additionally, under continuous exposure to Enzastaurin beginning 1, 2, 3, 4, 6, and 12 hours prior to 24 hours of Venetoclax treatment and continued Enastaurin treatment, the greatest efficacy of Venetoclax was demonstrated to occur with pre-dosing of Enzastaurin between 3-6 hours prior to Venetoclax treatment (FIG. 3B), compared to the reduced chemosensitisation to Venetoclax for Enzastaurin pre-dosing occurring before or after the 3-6 hour pre-treatment timeframe.

### Example 2—Inhibition of Stromal PKC-β Mitigates Environment-Mediated Drug Resistance

Approximately 20,000 PKC-β WT or PKC-β KO bone marrow derived stroma cells were seeded into tissue culture wells. Subsequently approximately 200,000 CLL patient cells were seeded for monoculture or co-culture with the PKC-β WT or PKC-β KO cells. 24 hours thereafter cultures were exposed to increasing doses of Venetoclax (BCL2-inhibitor), Bendamustine (alkylating agent), Fludarabine (purine analogue), Ibrutinib or Idelalisib (inhibitors of B-cell receptor-induced kinases). Cultures were subsequently evaluated for viability using flow cytometric analysis.

Results

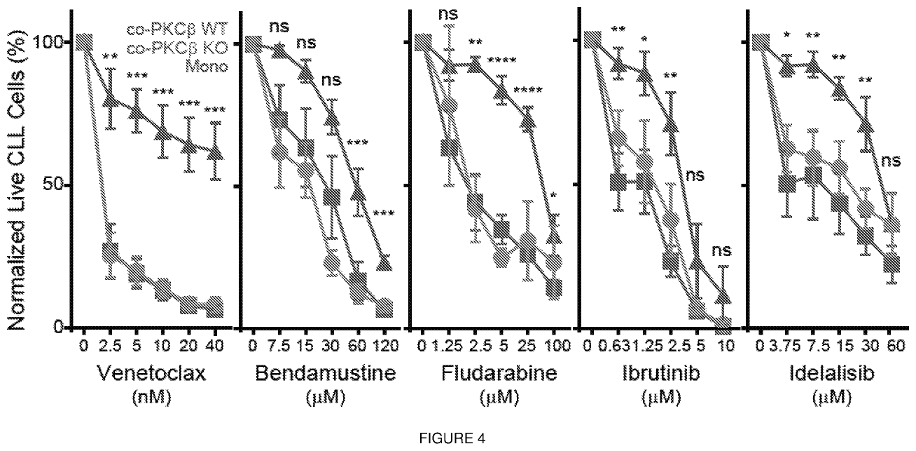

Co-culture with PKC-β WT cells significantly enhanced the resistance of CLL cells to the cytotoxic agents when compared to CLL cells in monoculture. In particular, strong protective effects of PKC-β WT cells were observed on CLL cells for Venetoclax and Fludarabine treatments. These protective effects were abolished or decreased in CLL cell co-culture with PKC-β KO cells under all treatments (FIG. 4).

### Example 3—PKC-8-inhibitors act synergistically with Venetoclax

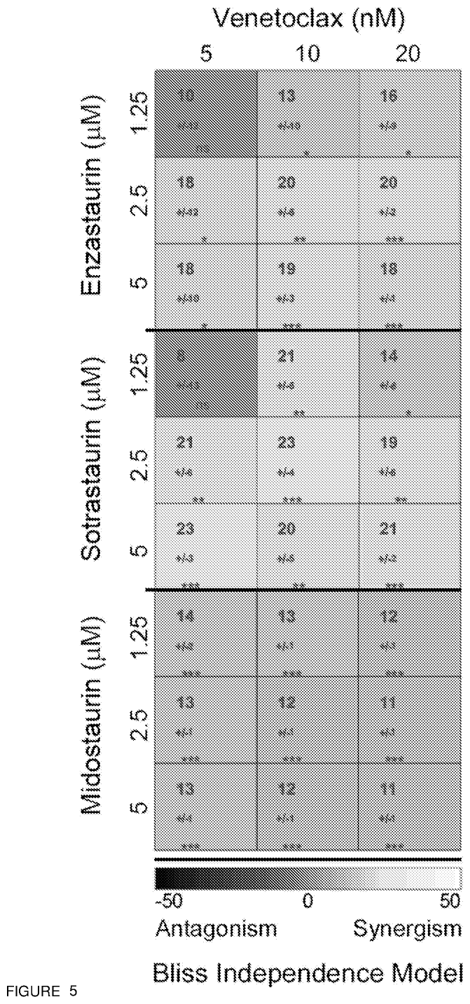

Approximately 20,000 wild-type bone marrow derived stroma cells were seeded into tissue culture wells and subsequently co-cultured with approximately 200,000 CLL patient cells. 24 hours thereafter, co-cultures were treated with Enzastaurin, Sotrastaurin or Midostaurin (1.25 μM, 2.5 μM, 5 μM) prior to treatment with Venetoclax. Co-cultures were subsequently treated with Venetoclax (5 nM, 10 nM, 20 nM) for 24 hours. Cells were subsequently evaluated for viability using flow cytometric analysis. Synergism was calculated using Compusyn Software (CRUK), within the Bliss Independence model (FIG. 5).

Results

Combinatorial treatment with Venetoclax and Enzastaurin, Sotrastaurin or Midostaurin produced synergistic effects, showing that PKC-β-inhibitors can chemosensitize malignant B cells to cytotoxic agents.

### Example 4—PKC-f Expression in MSCs is Essential for Normal B1 Cell Development

Generation of Chimeric Mice.

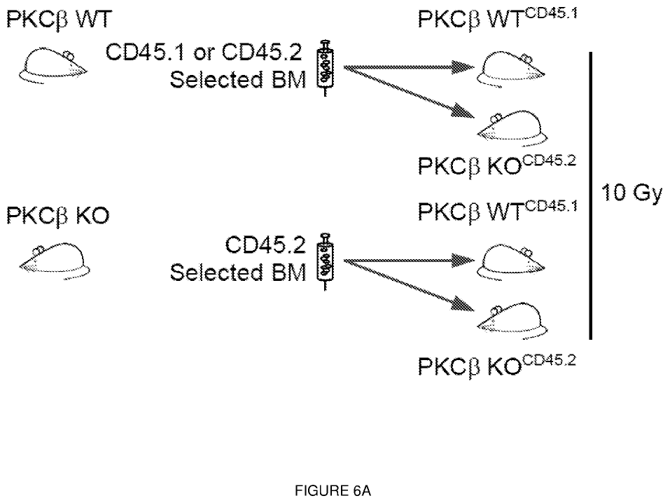

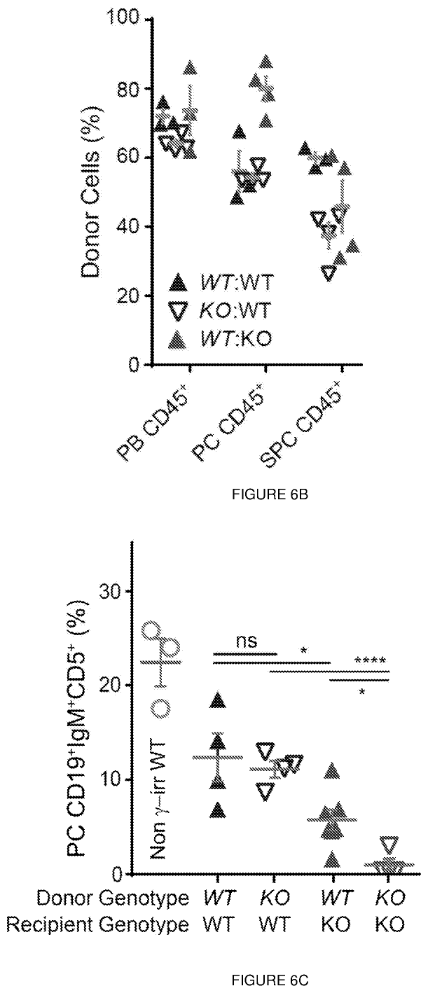

Bone marrow from CD45.2⁺ Prkcb^{+/+}, Prkcb^{-/-}, and CD45.1+B6.SJL-Ptprca Pepcb/BoyJ (Jackson Labs, USA) age-matched mice were isolated and depleted of CD45⁻ cells with purity of >95% confirmed by flow cytometry (muCD45 microbeads; Miltenyi Biotec). 3*10⁶ cells of purified bone marrow of respective CD45.2⁺ Prkcb^{-/-} purified-BM and CD45.1⁺ B6.SJL-Ptprca Pepcb/BoyJ purified-BM were injected intravenously into respectively different CD45 recipients, post-irradiation (10 Gy) (i.e. CD45.1⁺ BM into CD45.2 recipient and CD45.2⁺ BM into CD45.1 recipient). CD45.2⁺ Prkcb^{-/-} BM was also injected into irradiated CD45.2⁺ Prkcb^{-/-} recipients as a control (FIG. 6A). Chimerism was assessed by flow cytometry of CD45.1 and CD45.2 staining of peripheral blood withdrawn by tail vein bleeding (FIG. 6B).

The inventors investigated whether the lack of tumor cell engraftment in PKC-β KO mice was entirely attributed to its absence in MSCs or whether hematopoietic cells in the microenvironment also contributed. By generating mixed chimera, differing only in the expression of PKC-β in the hematopoietic system, the inventors also addressed whether the engraftment-dependence on microenvironmental PKC-β signals reflects properties of the cell-of-origin. The cell-of-origin is thought to be a CD5+B cell in mouse and man, in mouse most likely a CD5+B1 cell, an innate type of B cell responsible for the production of natural antibodies. The inventors generated PKC-β chimeric mice by transplanting PKC-β WT CD45⁺ hematopoietic bone marrow cells into irradiated (10 Gy) KO animals (as described above). To allow for the assessment of chimerism WT CD45.1⁺ bone marrow cells were transplanted into CD45.2+KO recipient mice. As controls, KO CD45.2+BM cells were transplanted into CD45.1+WT recipient mice (FIG. 6A). 10 weeks post transplantation, a mixed chimerism was observed in the peripheral blood with a predominance of the transplanted bone marrow cells (WT(donor):WT(recipient)=72.0% *2.09%, WT:KO=73.7±7.11%, KO:WT=64.1%±1.13%; FIG. 6B). Germ-line deletion of PKC-β in mice causes immunodeficiency with a marked reduction of peritoneal B1 cells and a significant reduction in serum IgM and IgG3 (Leitges et al., 1996). Strikingly, in WT recipient animals the inventors found no difference in the number of peritoneal B1 cells derived from either PKC-β KO or WT donor cells. Conversely, the development of peritoneal B1 cells in KO recipient animals transplanted from WT bone marrow was significantly reduced compared to WT recipient animals. Notably, the number of peritoneal B1 cells was still higher than in PKC-β KO control recipients reconstituted with KO bone marrow (FIG. 6C). These data demonstrate that PKC-β is an important cell-extrinsic factor for B cell development.

### Example 5—Pre-Treatment with Enzastaurin Increases the efficacy of Bendamustine

1-Hour Exposure to Enzastaurin

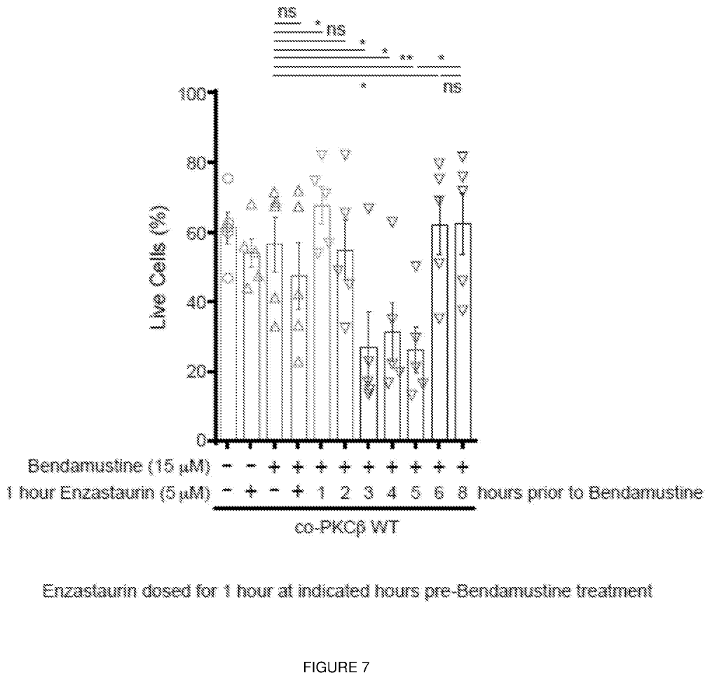

Approximately 20,000 wild-type bone marrow derived stroma cells were seeded into tissue culture wells and subsequently co-cultured with approximately 200,000 CLL patient cells. 24 hours thereafter, co-cultures were treated with 5 μM Enzastaurin for 1 hour at the indicated time-points prior to treatment with Bendamustine (FIG. 7). Equivalent PKC-β-inhibitor exposure was maintained through removal and subsequent washout of Enzastaurin-treated co-cultures. Co-cultures were centrifuged at 500 g for 5 minutes, prior to removal of Enzastaurin containing media. Washout with cell culture media, was followed by an additional centrifugation at 500 g for 5 minutes, and the replacement of washout media with fresh media. Respective co-cultures were subsequently treated with Bendamustine (15 μM) for 24 hours. Cells were subsequently evaluated for viability using Annexin-V-FITC and DAPI, or 4′,6-diamidino-2-phenylindole, staining on a flow cytometer.

Pre-treatment with equivalent Enzastaurin exposure showed increased efficacy for Bendamustine at pre-dosing timepoints of 2-6 hours, with the largest effect being observed at 3-6 hours (FIG. 7).

Continuous Exposure to Enzastaurin

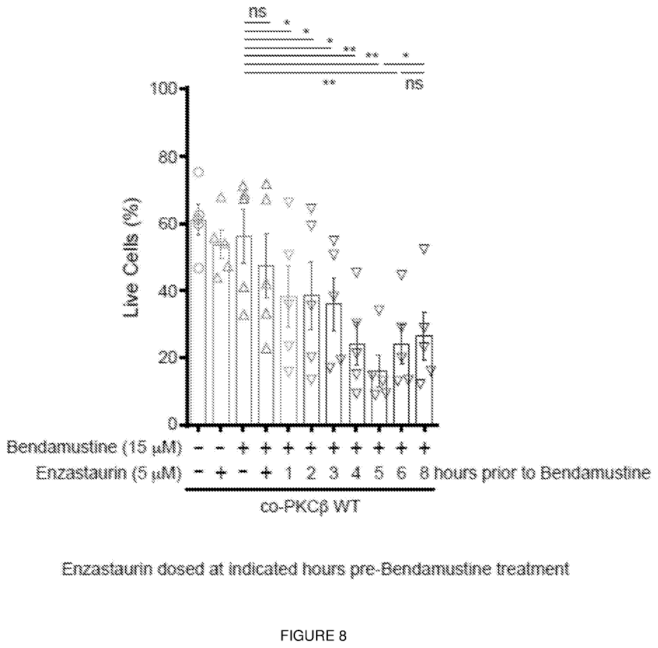

Approximately 20,000 wild-type bone marrow derived stroma cells were seeded into tissue culture wells and subsequently co-cultured with approximately 200,000 CLL patient cells. 24 hours thereafter, co-cultures were treated with 5 μM Enzastaurin at the indicated time-points prior to treatment with Bendamustine (FIG. 8). Co-cultures were subsequently treated with Bendamustine (15 μM) for 24 hours. Cells were subsequently evaluated for viability using Annexin-V-FITC and DAPI staining on a flow cytometer (FIG. 8).

Under continuous exposure to Enzastaurin beginning 1, 2, 3, 4, 6, and 8 hours prior to 24 hours of Bendamustine treatment and continued Enzastaurin treatment, the greatest efficacy of Bendamustine was demonstrated to occur with pre-dosing of Enzastaurin between 3-6 hours prior to Bendamustine treatment (FIG. 8), compared to the reduced chemosensitisation to Bendamustine for Enzastaurin pre-dosing occurring before or after the 3-6 hour pre-treatment timeframe.

### Example 6—Pre-treatment with Midostaurin increases the efficacy of Bendamustine

Continuous Exposure to Midostaurin

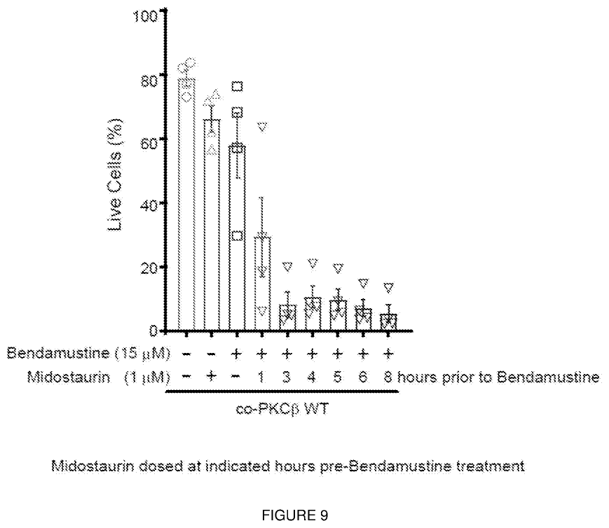

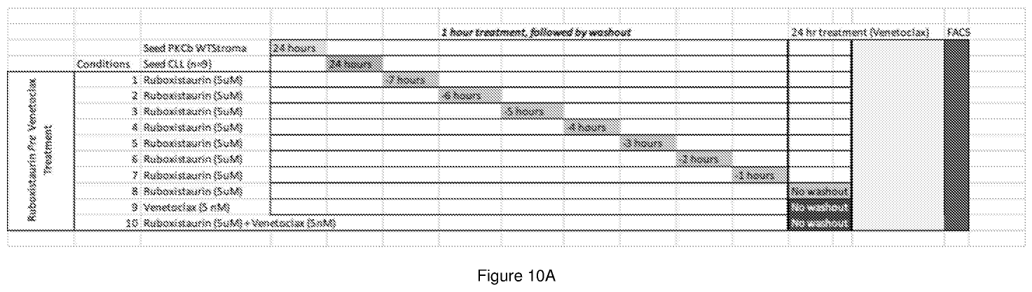

Approximately 20,000 wild-type bone marrow derived stroma cells were seeded into tissue culture wells and subsequently co-cultured with approximately 200,000 CLL patient cells. 24 hours thereafter, co-cultures were treated with 1 μM Midostaurin at the indicated time-points prior to treatment with Bendamustine (FIG. 8). Co-cultures were subsequently treated with Bendamustine (15 μM) for 24 hours. Cells were subsequently evaluated for viability using Annexin-V-FITC and DAPI staining on a flow cytometer (FIG. 9).

Under continuous exposure to Midostaurin beginning 1, 2, 3, 4, 6, and 8 hours prior to 24 hours of Bendamustine treatment and continued Midostaurin treatment, the greatest efficacy of Bendamustine was demonstrated to occur with pre-dosing of Midostaurin 3-8 hours prior to Bendamustine treatment (FIG. 9), compared to the reduced chemosensitisation to Bendamustine for Midostaurin pre-dosing occurring before the 3 hour pre-treatment timeframe.

Example 7—Pre-Treatment with Ruboxistaurin Increases the Efficacy of Venetoclax

1-Hour Exposure to Ruboxistaurin

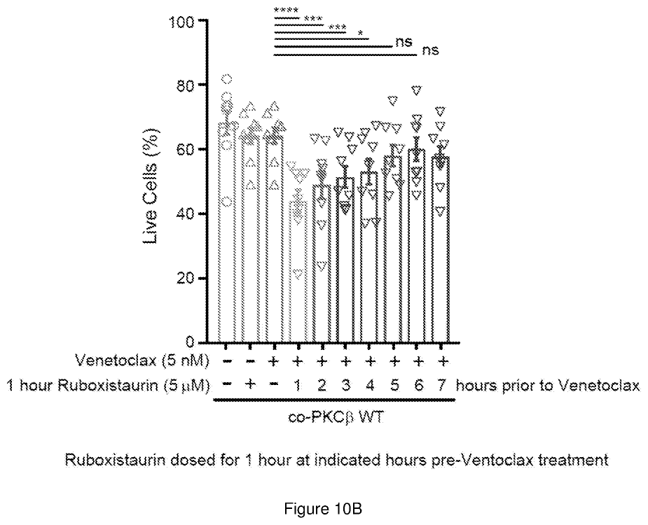

Approximately 20,000 wild-type bone marrow derived stroma cells were seeded into tissue culture wells and subsequently co-cultured with approximately 200,000 CLL patient cells. 24 hours thereafter, co-cultures were treated with 5 μM Ruboxistaurin for 1 hour at the indicated time-points prior to treatment with Venetoclax (FIG. 10A). Equivalent PKC-β-inhibitor exposure was maintained through removal and subsequent washout of Ruboxistaurin-treated co-cultures. Co-cultures were centrifuged at 500 g for 5 minutes, prior to removal of Ruboxistaurin containing media. Washout with cell culture media, was followed by an additional centrifugation at 500 g for 5 minutes, and the replacement of washout media with fresh media. Respective co-cultures were subsequently treated with Venetoclax (5 nM) for 24 hours. Cells were subsequently evaluated for viability using Annexin-V-FITC and DAPI, or 4′,6-diamidino-2-phenylindole, staining on a flow cytometer (FIG. 10B).

### Results

Pre-treatment with Ruboxistaurin exposure showed increased efficacy for Venetoclax at pre-dosing time points of 1-6 hours with the most significant effects being observed between 1-4 hours (FIG. 10B).

### REFERENCES

Ahn et al. (2017) Blood 129, 1469-1479
Chapuy et al. (2018) Nat. Med. 24, 679-690.
Dave et al. (2004) N. Engl. J. Med. 351, 2159-2169.
de Rooij et al. (2012) Blood 119, 2590-2594.
El Fassi et al. (2007) J. Clin. Endocrinol. Metab. 92, 1769-1772.
Gladstone et al. (2006) Arch Neurol. 63, 1388-1393.
Hampe, (2012) Scientifica 2012, 215308.
Herman et al. (2015) Clin. Cancer Res. 21,4642-4651.
Hofmann et al., (2018) Front Immunol. 9, 835.
Ramos-Casals et al. (2008) Medicine 87,354-364.
Ramsay et al. (2012) Blood 120, 1412-1421.
Leitges et al. (1996) Science 273, 788-791.
Lenz et al. (2008) N. Engl. J. Med. 359 2313-2323.
Lutzny et al. (2013) Cancer Cell 23, 77-92
Makhani et al. (2009) Neurology 72, 2076-2082.
Martin & Chan (2004) Immunity 20,517-527.
Puente et al. (2011) Nature 475, 101-105.
Quesada et al. (2012) Nat. Genet. 44,47-52
Schmitz et al. (2018) N. Engl. J. Med. 3781396-1407.
St. Clair et al. (2004) Arthritis Rheum. 50,3432-3443.
Xu-Monette et al. (2018) Blood 131, 68-83.
Wang et al. (2015) J. Intern. Med. 278,369-395.

1. A method for increasing the sensitivity of a subject to a cytotoxic agent, the method comprising administering a PKC inhibitor and a cytotoxic agent to the subject, wherein the PKC inhibitor reaches a peak concentration in the subject prior to the cytotoxic agent reaching a peak concentration.

2. A method for treating cancer or an autoimmune disease in a subject, the method comprising administering a PKC inhibitor and a cytotoxic agent to the subject, wherein the PKC inhibitor reaches a peak concentration in the subject prior to the cytotoxic agent reaching a peak concentration.

3. The method of claim 2, wherein the method is for treating cancer.

4. The method of claim 3, wherein the cancer is selected from the group consisting of lymphoma, leukemia, breast cancer, bile duct cancer, bladder cancer, gastric cancer, lung cancer, prostate cancer, colon cancer and colorectal cancer.

5. The method of claim 4, wherein the cancer is selected from the group consisting of chronic lymphocytic leukemia (CLL), mantle cell lymphoma (MCL), acute lymphoblastic leukemia (B-ALL) and acute myeloid leukemia (AML), follicular lymphoma, diffuse large B cell lymphoma and Burkitt lymphoma.

6. The method of claim 2, wherein the method is for treating the autoimmune disease.

7. The method of claim 6, wherein the autoimmune disease is selected from the group consisting of rheumatoid arthritis, systemic lupus erythematosus, inflammatory bowel disease, multiple sclerosis, diabetes mellitus type 1, celiac disease, Grave's disease, psoriasis, and vasculitis.

8. The method of claim 2, wherein the peak concentration of the PKC inhibitor is the maximum concentration that the PKC inhibitor reaches in the blood, cerebrospinal fluid, a target organ or a tumour after administration to the subject and the peak concentration of the cytotoxic agent is the maximum concentration that the cytotoxic agent reaches in the blood, cerebrospinal fluid, the target organ or the tumour after administration to the subject.

9. The method of claim 2, wherein the PKC inhibitor is a PKC-β inhibitor.

10. The method of claim 2, wherein the PKC inhibitor is enzastaurin, sotrastaurin, midostaurin, ruboxistaurin, or a pharmaceutically acceptable salt or solvate thereof.

11. The method of claim 10, wherein the PKC inhibitor is enzastaurin, or the pharmaceutically acceptable salt or solvate thereof.

12. The meth of claim 10, wherein the PKC inhibitor is ruboxistaurin, or the pharmaceutically acceptable salt or solvate thereof.

13. The method of claim 2, wherein the cytotoxic agent is a mitosis inhibitor, a nucleoside analogue, an anthracycline, a DNA-intercalating agent, an alkylating agent, an antimetabolite, an anti-microtubule agent, a folate antagonist, a topoisomerase inhibitor, an apoptosis inducer, a BCL-2 inhibitor, a BTK inhibitor, a P3K inhibitor, a glucocorticoid, or a cytotoxic antibody.

14. The method of claim 2, wherein the cytotoxic agent is fludarabine, venetoclax, methotraxate, vincristine, dexamethasone, an anthracycline, bendamustine, idealisib, ibrutinib, methotrexate, cyclophosphamide, a steroid or a monoclonal antibody targeting B cells, or a pharmaceutically acceptable salt or solvate thereof.

15. The method of claim 14, wherein the cytotoxic agent is fludarabine, venetoclax, bendamustine, or the pharmaceutically acceptable salt or solvate thereof.

16. The method of claim 2, wherein the PKC inhibitor reaches the peak concentration in the subject between 30 minutes and 12 hours prior to the cytotoxic agent reaching the peak concentration.

17. The method of claim 16, wherein the PKC inhibitor reaches the peak concentration in the subject between 1 and 8 hours or between 2 and 6 hours prior to the cytotoxic agent reaching the peak concentration.

18. The method of claim 17, wherein the PKC inhibitor reaches the peak concentration in the subject between 3 and 5 hours prior to the cytotoxic agent reaching the peak concentration.

19. A pharmaceutical composition, the composition comprising a PKC inhibitor and a cytotoxic agent, or a pharmaceutically acceptable salt or solvate thereof, and a pharmaceutically acceptable vehicle.

20. A process for making the composition of claim 19, the process comprising contacting a therapeutically effective amount of a PKC inhibitor, or a pharmaceutically acceptable salt or solvate thereof, a cytotoxic agent, or a pharmaceutically acceptable salt or solvate thereof, and a pharmaceutically acceptable vehicle.
